(19) United States
(12) Patent Application Publication
(10) Pub. No.: US 2025/0114159 A1
(43) Pub. Date: Apr. 10, 2025

(54) DEVICE FOR ADJUSTING MOVEMENT DISTANCE OF REMOTE SURGERY END EFFECTOR, AND METHOD THEREFOR

(71) Applicant: ROEN Surgical, Inc., Daejeon (KR)

(72) Inventors: Un-Je YANG, Daejeon (KR); Jong-Seok WON, Gyeonggi-do (KR); Duk-Sang KIM, Daejeon (KR)

(73) Assignee: ROEN Surgical, Inc., Daejeon (KR)

(21) Appl. No.: 18/293,644

(22) PCT Filed: Aug. 1, 2022
(86) PCT No.: PCT/KR2022/011264

§ 371 Date: Apr. 19, 2024

(30) Foreign Application Priority Data
Aug. 2, 2021 (KR) 10-2021-0101662

(51) Int. Cl. A61B 34/35; A61B 34/00

(52) U.S. Cl. CPC A61B 34/35; A61B 34/74

(57) ABSTRACT

The present invention relates to a device for adjusting the movement distance of a remote surgery end effector, and a method therefor, and more particularly, to a device for adjusting the movement distance of a remote surgery end effector, and a method therefor, the device being capable of changing the operating scale of an end effector according to screen magnification or, oppositely, changing the screen magnification according to operating scale. To this end, the device for adjusting the movement distance of a remote surgery end effector includes: an input device unit for generating an manipulation command of an end effector according to manipulation by a user in a remote place; a screen magnification control unit for decreasing or increasing the screen magnification of a captured image in conjunction with the adjustment of a first operating scale of the end effector according to a screen magnification change input or an operation mode change; an operating scale control unit for adjusting the first operating scale of the end effector in conjunction with a decrease and an increase in screen magnification, and transmitting, to the screen magnification control unit, the first operating scale of the end effector converted according to an operating scale change input or an operation mode change; and an end effector driving unit for changing the movement distance of the end effector according to the first operating scale.

### TECHNICAL FIELD

The present invention relates to a device for adjusting the movement distance of a remote surgery end effector, and a method therefor, and more particularly, to a device for adjusting the movement distance of a remote surgery end effector which converts an operation scale of the end effector according to a screen magnification or converts a screen magnification according to the operation scale, and a method therefor.

### BACKGROUND ART

Korean Patent Application Publication No. KR 10-2012-0068597 discloses a technique for adjusting a moving scale factor by decreasing the moving scale factor or increasing the moving scale factor according to an image photographing magnification. Such a known technology has an advantage in that a scale factor is automatically adjusted according to a high magnification or a low magnification of a screen magnification, thereby enabling detailed operation of an end effector (or a surgical tool).

However, the prior patent document discloses only a technique in which a scale factor is adjusted according to a screen magnification (hereinafter, referred to as the first mode), but does not disclose a technique in which a screen magnification is adjusted according to the adjustment of a scale factor (hereinafter, referred to as the second mode), and furthermore, does not disclose a technique of how to operate the end effector in harmony with the operation of the first and second modes.

### DISCLOSURE

### Technical Problem

Accordingly, the present invention has been made keeping in mind the above problems occurring in the prior art, and an objective of the present invention is to provide a device and a method for controlling a screen magnification, which can change/convert an operation scale of an end effector in connection with a screen magnification, or can change a screen magnification in connection with an operation scale of an end effector.

However, the objects of the present invention are not limited to the above-mentioned objectives, and other objectives not mentioned may be clearly understood by the skilled person in the art from the following description.

### Technical Solution

According to an aspect of the present invention, there is provided a device for adjusting the movement distance of a remote surgical end effector, the device including: an input device unit configured to generate an operation command of the end effector according to an operation of a user in a remote place; a screen magnification control unit configured to increase or decrease the screen magnification of a captured image in conjunction with adjustment of the first operation scale of the end effector according to a screen magnification change input or an operation mode change; an operation scale control unit configured to adjust the first operation scale of the end effector in conjunction with increase or decrease of the screen magnification, and transmit the first operation scale of the end effector, which is converted according to the operation scale change input or the operation mode change, to the screen magnification control unit; and an end effector driving unit configured to change the movement distance of the end effector according to the first operation scale.

The input device unit includes:
an operation command generator for r generating an operation command of the end effector;
an input device operation scale adjustment unit for adjusting the second operation scale of end effector
and an input device operation scale change unit, which converts, according to the second operation scale, an operation scale of an end effector which is limited according to the first operation scale to transmit to end effector driving unit.

Also, the input device unit further includes the first operation mode control unit for controlling the first operation scale to be adjusted in conjunction with the increase or decrease of a screen magnification while increasing/decreasing the screen magnification according to the first operation mode when a screen magnification change is operated;
and the operation mode control unit including the second operation mode control unit which controls the screen magnification to be adjusted in conjunction with the increase or decrease of the first operation scale according to the second operation mode while increasing or decreasing the first operation scale when the operation scale is changed.

The first operation mode control unit
transmits a magnification adjustment value according to the first operation mode to control the first operation scale to be adjusted in conjunction with the increase or decrease of the screen magnification,
and the second operation mode control unit
transmits the first operation scale adjustment value according to the second operation mode thereby controlling to adjust screen magnification in conjunction with the increase or decrease of the first operation scale.

The device further includes a Change ratio setting unit to set a Change ratio of the first operation scale to the magnification adjustment value and a screen Change ratio to the first operation scale adjustment value.

Meanwhile, the present invention has been made in an effort to provide a method for adjusting the movement distance of remote surgical end effector, which includes the steps of: adjusting an increase/decrease in screen magnification; decreasing the first operation scale by being interlocked with a Change ratio when the screen magnification is expanded, and increasing the first operation scale by being interlocked with the Change ratio when the screen magnification is decreased; primarily changing an operation effector distance of the end according to the increase/decrease in the first operation scale; and secondarily changing the operating distance of the end effector by changing the operating scale of the limited end effector according to the first operation scale according to the adjustment of the increase/decrease in the second operation scale.

Meanwhile, the objective of the present invention may be achieved by providing a method for adjusting the movement distance of remote surgical end effector including: adjusting an increase/decrease in the first operation scale; decreasing a screen magnification by being linked to a Change ratio when the first operation scale increases, and increasing the screen magnification by being linked to the Change ratio when the first operation scale decreases; primarily changing an operation distance of the end effector according to the increase/decrease in the first operation scale; and secondarily changing the operating distance of the end effector by changing the operating scale of the end effector limited according to the first operation scale according to the adjustment of the increase/decrease in the second operation scale.

### Advantageous Effects

According to the present invention as described above, the screen magnification can be changed in accordance with the operation scale of the end effector, or can be changed in accordance with the operation scale of the end effector.

### DESCRIPTION OF DRAWINGS

The following drawings attached to the present specification exemplify a preferred embodiment of the present invention and serve to further understand the technical spirit of the present invention together with the detailed description of the present invention, and thus the present invention should not be construed as being limited to the matters described in such drawings.

FIG. 1 is a diagram illustrating an overall feature of a device for adjusting the movement distance of a remote surgical end effector according to an embodiment of the present invention,

FIG. 2 is a diagram illustrating that the end effector is driven in the first operation mode and the second operation mode under control of an operation mode control unit according to an embodiment of the present invention,

FIG. 3 is a diagram illustrating that an operation scale is adjusted in conjunction with adjustment of an image magnification according to an embodiment of the present invention,

FIG. 4 is a diagram illustrating that an operation scale is adjusted in conjunction with adjustment of an operation scale according to an embodiment of the present invention,

FIGS. 5 and 6 are diagrams illustrating that an operation distance of the end effector may be changed without being limited to an operation scale changed by an operation scale control unit according to FIGS. 3 and 4 when an operation scale is changed by a master input device according to an embodiment of the present invention,

FIG. 7 is a diagram illustrating an actual movement distance of the end effector according to an increase in an image according to an embodiment of the present invention,

FIG. 8 is a diagram illustrating an actual movement of an end effector according to distance increasing/decreasing the screen magnification according to an embodiment of the present invention,

FIG. 9 is a diagram illustrating a method of increasing/decreasing an operation scale in conjunction with an increase/decrease in a screen magnification according to an embodiment of the present invention, and

FIG. 10 is a diagram illustrating a method of increasing/decreasing the screen magnification in conjunction with an increase/decrease in an operation scale according to an embodiment of the present invention.

### MODE FOR INVENTION

Hereinafter, a preferred embodiment of the present invention will be described with reference to the drawings. In addition, the exemplary embodiment described below does not limit the contents of the present invention described in the claims, and it cannot be said that the entire feature described in the present embodiment is essential as a means for solving the present invention. Further, descriptions of the prior art and the skilled person in the art may be omitted, and descriptions of the omitted elements (methods) and functions may be fully referred to without departing from the technical spirit of the present invention.

### (A Feature and a Function of a Device for Adjusting the Movement Distance of a Remote Surgical End Effector)

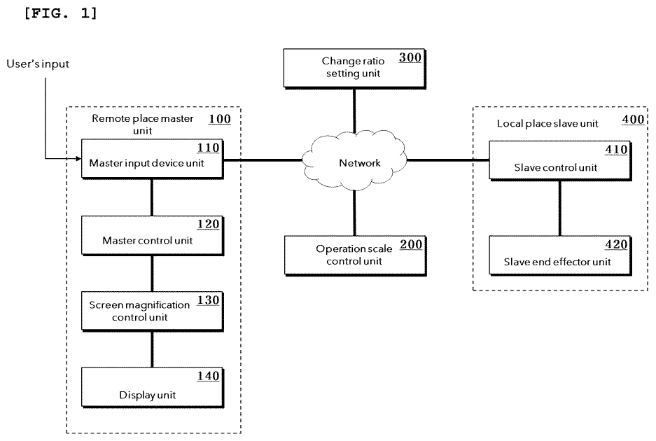

As shown in FIG. 1, the device for adjusting the movement distance of a remote surgical end effector includes a remote place master unit 100 and a local place slave unit 400. The remote place master unit 100 includes a device for the user to control the surgical robot of the local place slave unit 400 at a remote location. In addition, the local place slave unit 400 includes a surgical robot or the like that performs a driving operation according to a control command of the remote place master unit 100 at a local location.

To this end, as shown in FIG. 1, the remote place master unit 100 includes a master input device unit 110, a master control unit 120, and a screen magnification control unit 130. The local place slave unit 400 includes a slave control unit 410 and a slave end effector unit 420. Further, the local place slave unit includes the operation scale control unit 200 and the Change ratio setting unit 300, which convert or readjust the operation scale of the end-effector and transmit the converted or readjusted operation scale before the operation distance of the local end-effector, which is input by the user at a remote location, is transmitted to the local place slave unit 400. The operation scale control unit 200 and the Change ratio setting unit 300 convert the Movement distance or the operation distance (hereinafter, referred to as operation scale) of the local end effector according to a predetermined condition. Meanwhile, the remote place master unit 100 and the local place slave unit 400 may further include an additional feature for remotely controlling the surgical robot, etc. in addition to the above-described feature, and may be referred to within a range without departing from the technical spirit of the present invention.

The master input device unit 110 illustrated in FIG. 1 generates an operation command of an end effector according to a user's operation at a remote location. That is, the local end effector is driven according to the operation command considering the position and direction generated by the master input device unit 110. Therefore, the master input device unit 110 may be a 2D or 3D joystick capable of inputting a position and a direction, but is not limited thereto. In addition, the master input device unit 110 converts the operation scale of the end effector independently of the operation scale control unit 200. Therefore, in the present invention, in order to change the operation scale of the end effector, the first operation scale is adjusted by the operation scale control unit 200, and the second operation scale is readjusted by the master input device unit 110 independently of the operation scale control unit 200.

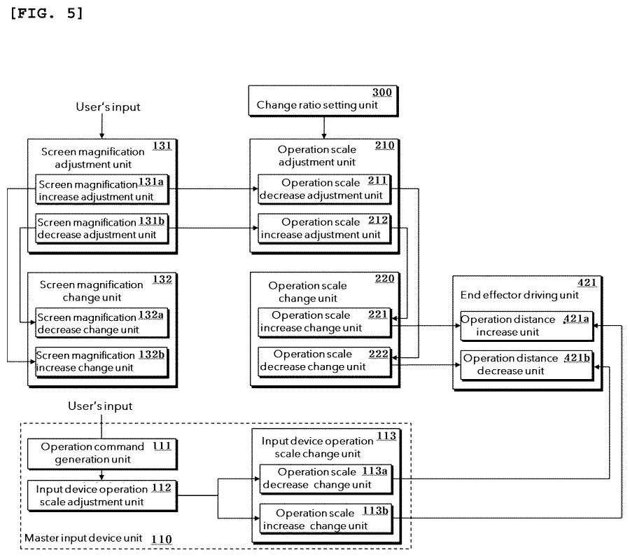

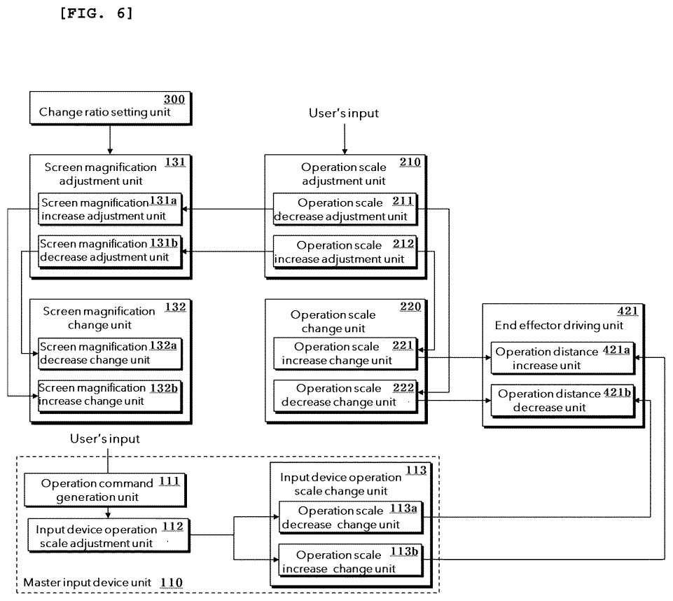

To this end, as shown in FIGS. 5 and 6, the master input device unit 110 includes an operation command generation unit 111, an input device operation scale adjustment unit 112, and an input device operation scale change unit 113.

As described above, the operation command generation unit 111 may be embodied as a two-dimensional or three-dimensional joystick. Accordingly, the position and direction for driving the end effector are input by the user to the operation command generation unit 111, and an operation command is generated accordingly.

The input device operation scale adjustment unit 112 receives an operation scale adjustment value from a user and adjusts the second operation scale of the end effector. The second operation scale is a value by which the operation scale of the end effector is readjusted according to a value input by the user. Unlike this, as will be described later, the first operation scale is a value that is interlocked according to an adjustment value of the screen magnification and adjusts the operation scale of the end effector.

The input device operation scale change unit 113 changes the operation scale of the end effector (e.g., changes the operation scale according to the Change ratio 1:2) according to the second operation scale adjustment value (e.g., the Change ratio 1:2) transmitted from the input device operation scale adjustment unit 112, and transmits the changed operation scale to the end effector driving unit 421. That is, the second operation scale is a value obtained by readjusting the operation scale of the end effector limited according to the first operation scale. Therefore, the movement of the end effector limited according to the first operation scale may be changed by the readjustment of the second operation scale. For example, even when the scale is adjusted to 1:2 by the first operation scale, the operation scale may be independently adjusted so that the end effector may move less or more than 1:2 by the second operation scale.

The master control unit 120 shown in FIG. 1 controls various devices including a master input device unit 110, a screen magnification control unit 130, and a display unit 140 provided in the master. The slave control unit 410 controls various devices including the slave end effector unit 420 provided in the local area.

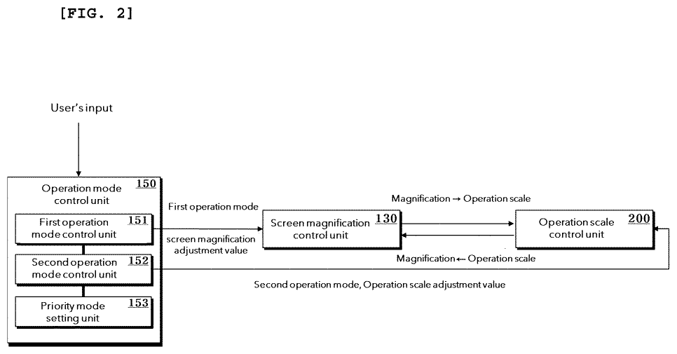

As shown in FIG. 2, the operation mode control unit 150 includes the first operation mode control unit 151 and the second operation mode control unit 152.

The first operation mode control unit 151 controls the first operation scale to be adjusted according to the first operation mode in association with the increase or decrease of the screen magnification when the user operates the magnification change of the photographing screen through the screen magnification control unit 130. That is, the first operation mode control unit 151 adjusts the first operation scale in conjunction with the change in the magnification of the screen. Therefore, when the magnification of the screen increases, the operation scale decreases, and when the magnification of the screen decreases, the operation scale increases.

On the contrary, the second operation mode control unit 152 controls the screen magnification to be adjusted in conjunction with the increase or decrease of the first operation scale according to the second operation mode when the user operates the change of the operation scale through the operation scale control unit 200. That is, the second operation mode control unit 152 adjusts the screen magnification in conjunction with the change of the operation scale. Therefore, when the operation scale increases, the screen magnification decreases, and when the operation scale decreases, the screen magnification increases.

As described above, the operation mode control unit 150 may smoothly control each operation mode when the operation scale is changed according to the screen magnification or when the screen magnification is changed according to the operation scale. As described above, in the first and second exemplary embodiments to be described below, a priority mode may be determined by the operation mode control unit 150 as an example of a case in which a user input is simultaneously input to the screen magnification control unit 130 and the operation scale control unit 200.

As the first embodiment, the screen magnification may be changed by a user's input in a user input change mode, or may be changed in conjunction with an operation scale in the second operation mode. In this example, the priority mode setting unit 153 sets to preferentially operate one of two modes, that is, the user input change mode and the second operation mode.

As the second embodiment, there are two cases in which the operation scale is changed by a user's input in the user input change mode or is changed in association with the screen magnification in the first operation mode. In this example, the priority mode setting unit 153 sets to preferentially operate one of two modes, i.e., the user input change mode and the first operation mode. At the same time, when no user input is made, the screen magnification may be linked to the screen magnification to readjust the operation scale, and then the screen magnification may be linked to the screen magnification to readjust the operation scale continuously. Alternatively, when a user input is not simultaneously performed, the screen magnification may be readjusted by interworking with the operation scale, and the operation scale may be readjusted by interworking with the operation scale by consecutively adjusting the screen magnification.

Meanwhile, the first operation mode control unit 151 transmits the magnification adjustment value input by the user according to the first operation mode to the screen magnification control unit 130 so that the first operation scale is adjusted in conjunction with the decrease or increase of the screen magnification. In addition, the second operation mode control unit 152 transmits the first operation scale adjustment value input from the user according to the second operation mode to the operation scale control unit 200 to control the screen magnification to be adjusted in conjunction with the increase or decrease of the first operation scale.

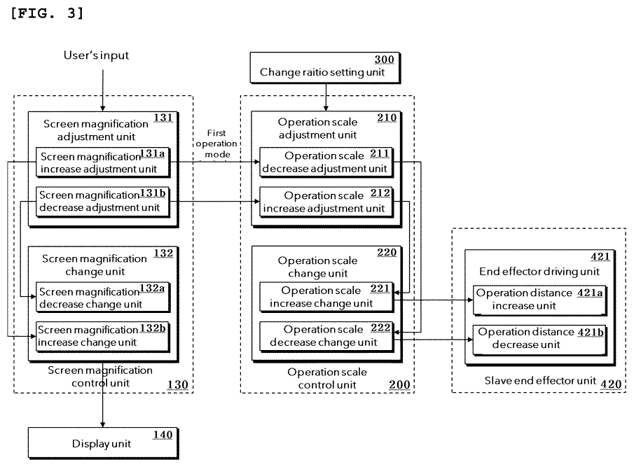

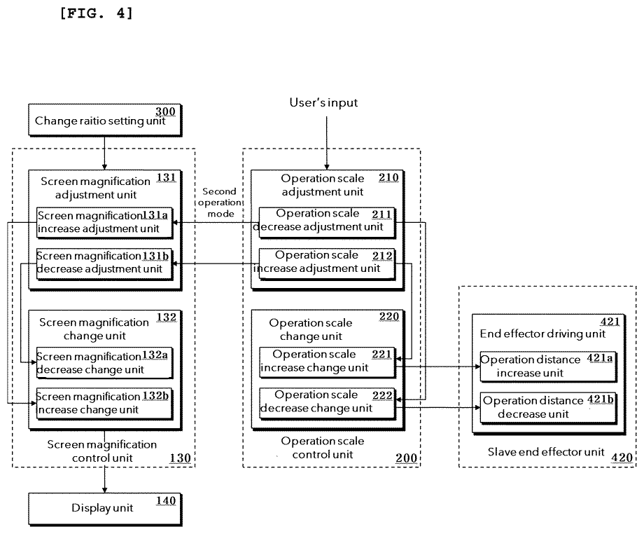

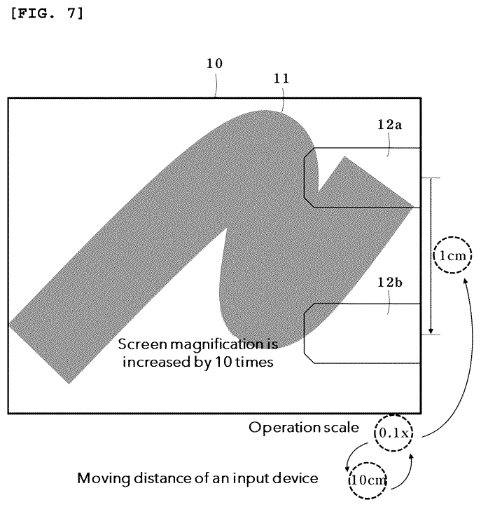

The Change ratio setting unit 300 shown in FIGS. 3 and 4 sets the first operation mode change ratio, which is a Change ratio of the first operation scale converted in conjunction with a magnification adjustment value. In addition, the second operation mode change ratio, which is a screen Change ratio converted in conjunction with the first operation scale adjustment value, is set. For example, as illustrated in FIG. 7, when the screen magnification is increased by 10 times based on the reference screen magnification, the operation scale is decreased by 0.1 times, and when the screen magnification is decreased by 10 times, the operation scale is increased by 10 times. As described above, the Change ratio setting unit 300 sets the first operation mode change rate, which is a change rate of the operation scale that is converted in conjunction with the screen magnification. In the same principle, the Change ratio setting unit 300 sets the second operation mode change rate that is a change rate of a screen magnification that is converted in conjunction with an operation scale. The first operation mode change ratio and the second operation mode change ratio may be set identically or differently. As an example, the first operation mode change ratio may be 10 times, and the second operation mode change ratio may be 5 times.

As shown in FIGS. 3 and 4, the screen magnification control unit 130 includes a screen magnification adjustment unit 131 and a screen magnification change unit 132. As shown in FIG. 2, the screen magnification control unit 130 may receive an adjustment value for the screen magnification from the operation mode control unit 150 or may directly receive an adjustment value for the screen magnification from the user.

As illustrated in FIG. 3, when a magnification adjustment command is input from the user, the screen magnification control unit 130 adjusts the screen magnification according to the increase/decrease command and displays the screen magnification on the display unit 140. In addition, the screen magnification control unit 130 transmits the screen magnification adjustment value to the operation scale control unit 200 in the first operation mode or the forward operation mode, so that the first operation scale may be adjusted and changed in conjunction with the screen magnification. In this case, the first operation scale value that is adjusted and changed is converted according to the first operation mode change rate value set by the Change ratio setting unit 300 described above.

Meanwhile, as shown in FIG. 4, the screen magnification control unit 130 may receive the first operation scale adjustment value from the operation scale control unit 200 in the second operation mode or the reverse operation mode to adjust the screen magnification.

As illustrated in FIGS. 3 and 4, the screen magnification adjustment unit 131 includes a screen magnification increase adjustment unit 131a and a screen magnification decrease adjustment unit 132b. The screen magnification increase adjustment unit 131a adjusts the screen magnification to increase. That is, the screen magnification increase adjustment unit 131a may be, for example, a jog dial or an electronic button for adjusting the screen magnification to increase or decrease by mechanically turning a dial or electronically pressing a button.

The screen magnification change unit 132 includes a screen magnification decrease change unit 132a and a screen magnification increase change unit 132b. The screen magnification decrease change unit 132a controls the screen magnification to be actually decreased and displayed on the monitor 140 according to the screen magnification decrease adjustment adjusted by the screen magnification decrease adjustment unit 131b. The screen magnification increase change unit 132b controls the screen magnification to actually increase and display on the monitor 140 according to the screen magnification increase adjustment adjusted by the screen magnification increase adjustment unit 131a.

The screen magnification adjustment unit 131 and the screen magnification change unit 132 are separately described for convenience of description, but may be integrated with each other.

As shown in FIGS. 3 and 4, the operation scale control unit 200 includes an operation scale adjustment unit 210 and an operation scale change unit 220. As shown in FIG. 2, the operation scale control unit 200 may receive an adjustment value for the operation scale from the operation mode control unit 150 or may directly receive an adjustment value for the operation scale from a user.

As shown in FIG. 3, the operation scale control unit 200 receives the screen magnification adjustment value according to the screen magnification and decrease from the screen magnification control unit 130 in the first operation mode or the forward operation mode, and adjusts and changes the first operation scale according to the first operation mode change ratio set by the Change ratio setting unit 300.

For example, when the transmitted screen magnification adjustment value is 10 times the screen magnification and the first operation mode change ratio is set to 1 times, the operation scale control unit 200 sets the operation scale to 0.1 times.

As another example, when the transmitted screen magnification adjustment value is 10 times the screen magnification and the first operation mode change ratio is set to 10 times, the operation scale control unit 200 sets the operation scale to 0.01 times.

Meanwhile, as shown in FIG. 4, the operation scale adjustment unit 200 transmits the first operation scale control value to the screen magnification control unit 130 in the second operation mode or the backward operation mode. The screen magnification control unit 130 adjusts and changes the screen magnification according to the received first operation scale adjustment value and the second operation mode setting ratio of the Change ratio setting unit 300. The example of the screen magnification change is the same as the above-described example, and will be replaced with the above-described example.

The operation scale adjustment unit 210 includes an operation scale decrease adjustment unit 211 and an operation scale increase adjustment unit 212. As shown in FIG. 3, the operation scale decrease adjustment unit 211 controls the first operation scale increase control value to be decreased according to the Change ratio set by the Change ratio setting unit 300 and the screen magnification increase control value transmitted in the first operation mode.

The operation scale increase adjustment unit 212 increases and adjusts the first operation scale adjustment value according to the Change ratio set by the Change ratio setting unit 300 and the screen magnification decrease adjustment value transmitted in the first operation mode. As shown in FIG. 4, in the second operation mode, the operation scale adjustment value is transmitted to the screen magnification adjustment unit 131.

The operation scale change unit 220 includes an operation scale increase change unit 221 and an operation scale decrease change unit 222. The operation scale increase change unit 221 generates a change increase value for converting the operation scale of the end effector according to the operation scale increase adjustment value adjusted by the operation scale increase adjustment unit 212, and transmits the change increase value to the end effector driving unit 421. The operation scale decrease change unit 222 generates a change decrease value for converting the operation scale of the end effector according to the operation scale decrease adjustment value adjusted by the operation scale decrease adjustment unit 211, and transmits the change decrease value to the end effector driving unit 421.

The above-described operation scale adjustment unit 210 and the operation scale change unit 220 are separately described for convenience of description, but may be integrated with each other.

Meanwhile, the operation distance increase unit 421a of the end effector driving unit 421 increases the movement distance of the end effector according to the transmitted change increase value. In addition, the operation distance decrease unit 421b of the end effector driving unit 421 decreases the movement distance of the end effector according to the transmitted change decrease value.

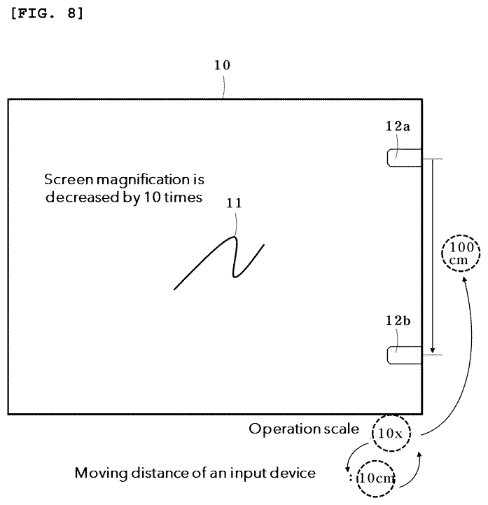

For example, as illustrated in FIGS. 7 and 8, a case where the movement distance of an input device by a user at a remote location is 10 cm will be described as an example. FIG. 7 illustrates a case where the screen is enlarged by 10 times, and when the screen is enlarged by 10 times, the operation scale is decreased by 0.1 times, and accordingly, the driving distance of the end effector is actually decreased to 1 cm. That is, although the input device operates to move the end effector 10 cm, the operation scale is adjusted according to the 10-fold enlargement of the screen, so that the end effectors 12a and 12b are actually moved by 1 cm.

In FIG. 8, the screen is decreased by 10 times, and when the screen is decreased by 10 times, the operation scale is increased by 10 times, and accordingly, the driving distance of the end effector is actually increased to 100 cm. That is, although the input device operates to move the end effector 10 cm, the operation scale is adjusted according to the decrease of the screen by 10 times, so that the end effectors 12a and 12b are actually moved 100 cm.

Of course, although not shown in the drawings, when the operation scale adjusted, is initially the screen magnification may be enlarged or decreased in conjunction with the adjustment of the operation scale in the same principle with reference to FIGS. 7 and 8.

### (A Method for Adjusting the Movement Distance of Remote Surgical End Effector)

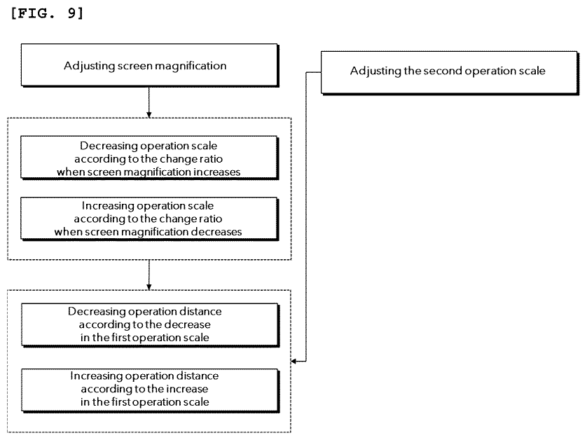

As illustrated in FIG. 9, an example in which an operation scale is adjusted in conjunction with an increase/decrease adjustment of a screen magnification will be described.

The increase or decrease in screen magnification for capturing organs or tissues is adjusted by the input by a user.

When the increases/decreases the screen in magnification is adjusted, the display device is linked to the Change ratio at the time of enlarging the screen magnification to decrease the first operation scale, and is linked to the Change ratio at the time of decreasing the screen magnification to increase the first operation scale.

According to the increase or decrease in the first operation scale, the movement distance of the end effector input to the input device by the user is primarily changed and moved.

As the increase/decrease in the second operation scale independently scale is controlled by the user from the increase/decrease in the first operation, the operation scale of the end effector limited according to the first operation scale is changed, thereby secondarily changing the operation distance of the end effector.

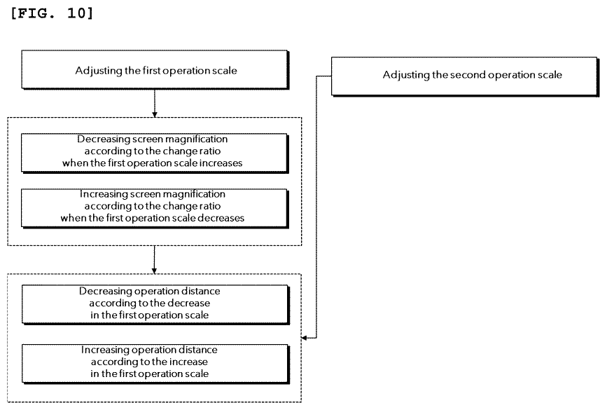

Meanwhile, as illustrated in FIG. 10, an example in which the screen magnification is adjusted in conjunction with the increase/decrease in the operation scale will be described.

An increase/decrease in the first operation scale is adjusted by a user input.

When the increase/decrease in the first operation scale is adjusted, the screen magnification is decreased by being interlocked according to the Change ratio when the first operation scale is increased, and the screen magnification is increased by being interlocked according to the Change ratio when the first operation scale is decreased. As the screen magnification increases/decreases in conjunction with the operation scale, the magnification displayed on the monitor is displayed by being adjusted in conjunction with the increase/decrease in the first operation scale.

According to the increase/decrease in the first operation scale, the movement distance of the end effector input to the input device by the user is primarily changed and moved.

As to the increase/decrease in the second operation scale is adjusted, the operation scale of the limited end effector is changed according to the first operation scale, and thus the operation distance of the end effector is secondarily changed.

In describing the present invention, descriptions of matters apparent to the prior art and the skilled person in the art may be omitted, and descriptions of elements (methods) and functions omitted may be fully referred to without departing from the technical spirit of the present invention. In addition, the above-described components of the present invention have been described for convenience of description of the present invention, but the components not described herein may be added within a range that does not deviate from the technical spirit of the present invention.

The description of the feature and the function of each of the above-described components is separately described for convenience of description, but any one feature and function may be integrated into other components or may be implemented in a more subdivided manner as necessary.

As described above, the present invention has been described with reference to an embodiment of the present invention, but the present invention is not limited thereto, and various modifications and applications are possible. That is, it will be easily understood by a skilled person in the art that many modifications are possible without departing from the gist of the present invention. In addition, it should be noted that when it is determined that a detailed description of a known function related to the present invention and a feature thereof or a coupling relation to each feature of the present invention may unnecessarily obscure the gist of the present invention, the detailed description thereof will be omitted.

1. A device for adjusting a movement distance of a remote surgical end effector, the device comprising:
an input device unit configured to generate an operation command of the end effector according to an operation of a user in a remote place;
a screen magnification control unit configured to increase or decrease a screen magnification of a captured image in conjunction with adjustment of a first operation scale of the end effector according to a screen magnification change input or an operation mode change;
an operation scale control unit configured to adjust the first operation scale of the end effector in conjunction with increase or decrease of a screen magnification, and transmit the first operation scale of the end effector, which is converted according to an operation scale change input or an operation mode change, to a screen magnification control unit; and
an end effector driving unit configured to change a movement distance of the end effector according to the first operation scale.

2. The device for adjusting a movement distance of a remote surgical end effector of claim 1, wherein the input device unit comprises:
an operation command generation unit configured to generate an operation command for the end effector;
an input device operation scale adjustment unit configured to adjust a second operation scale of the end effector; and
an input device operation scale change unit configured to change an operation scale of the end effector limited according to the first operation scale according to the second operation scale, and to transmit the changed operation scale to an end effector driving unit.

3. The device for adjusting a movement distance of a remote surgical end effector of claim 2, further comprising:
an operation mode control unit including:
a first operation mode control unit configured to control the first operation scale to be adjusted in conjunction with the increase or decrease of the screen magnification while increasing/decreasing the screen magnification according to a first operation mode when a screen magnification change is operated; and
a second operation mode control unit configured to control the screen magnification to be adjusted in conjunction with the increase or decrease of the first operation scale according to a second operation mode when an operation scale change is operated.

4. The device for adjusting a movement distance of a remote surgical end effector of claim 3, wherein the first operation mode control unit controls the first operation scale to be adjusted in conjunction with the increase or decrease of the screen magnification by transmitting a magnification adjustment value according to the first operation mode, and
the second operation mode control unit controls the screen magnification to be adjusted in conjunction with the increase or decrease of the first operation scale by transmitting a first operation scale adjustment value according to the second operation mode, as a feature.

5. The device for adjusting a movement distance of a remote surgical end effector of claim 1, further comprising:
a change ratio setting unit configured to set a change ratio of the first operation scale to a magnification adjustment value and a screen change ratio to the first operation scale adjustment value, wherein the feature is set as a change ratio setting unit.

6. A method for adjusting a movement distance of remote surgical end effector, the method comprising:
adjusting an increase/decrease in a magnification of a screen;
decreasing a first operation scale by interworking with a change ratio when the magnification of the screen is increased, and increasing the first operation scale by interworking with the change ratio when the magnification of the screen is decreased;
primarily changing an operation distance of the end effector according to the increase/decrease in the first operation scale; and
secondarily changing the operating distance of the end effector by changing a limited operating scale of the end effector according to the first operation scale according to the increase/decrease in a second operation scale.

7. A method for adjusting a movement distance of remote surgical end effector, the method comprising:
adjusting an increase/decrease of a first operation scale;
decreasing a screen magnification by being linked to a change ratio when the first operation scale increases, and increasing the screen magnification by being linked to the change ratio when the first operation scale decreases;
primarily changing an operation distance of the end effector according to the increase/decrease of the first operation scale; and
secondarily changing the operating distance of the end effector by changing a limited operating scale of the end effector according to the first operation scale when the increase/decrease of a second operation scale is adjusted, wherein the adjustment of the first operation scale and the adjustment of the second operation scale are features.
